(19) United States
(12) Patent Application Publication
(10) Pub. No.: US 2025/0032747 A1
(43) Pub. Date: Jan. 30, 2025

(54) Multi-Component Medical Packaging Device

(71) Applicant: Philip Meyers, Boise, ID (US)

(72) Inventor: Philip Meyers, Boise, ID (US)

(21) Appl. No.: 18/403,688

(22) Filed: Jan. 3, 2024

Related U.S. Application Data
(60) Provisional application No. 63/516,229, filed on Jul. 28, 2023.

(51) Int. Cl. A61M 25/00; A61M 25/09

(52) U.S. Cl. CPC A61M 25/002; A61M 25/09; A61M 2025/0042; A61M 2207/00

(57) ABSTRACT

Packaging that is meant to store and protect sterile catheter and guidewire assemblies for acute stroke treatment and other sterile vascular procedures. One end includes an access port for appropriately trained medical staff to expel gas, or “room air”, from the packaging under limited pressure, including catheter and guidewire assemblies, in a sterile fashion using a biocompatible liquid. Then, the medical personnel can release the catheter and guidewire assemblies from the packaging. With mild traction, the catheter and guidewire assemblies can be removed from the packaging in a slidable manner from another end of the packaging for immediate use in treatment of a hospitalized patient.

### REFERENCE TO RELATED APPLICATION

This application claims priority to U.S. Provisional Patent Application Ser. No. 63/516,229 filed Jul. 28, 2023; the disclosure of which is incorporated herein in its entirety.

### BACKGROUND

There are many vascular emergencies. Common examples of vascular emergencies are myocardial infarction due to coronary artery diseases and stroke due to cardiac and cerebral vascular diseases. In these emergency situations, and even in non-emergency situations, time is critical. In fact, according to current guidelines published by the American Heart Association and American Stroke Association and administered by The Joint Commission or other regulatory agency, a thrombectomy procedure should begin within ninety-minutes of a patient's arrival at a catheterization-capable stroke healthcare center. Clinical stroke trials have shown that a 30-minute delay in restoring brain blood flow causes a 10% reduction in a patient's chance for neurological recovery. For these reasons, “time is brain” has been a commonly used slogan to promote adoption of a rapid stroke triage and treatment paradigm. For a stroke center to meet the AHA guideline by diagnosing and evaluating the stroke patient for treatment is challenging. As such, every effort must be made to streamline every part of the triage system to optimize the time to treatment. In fact, some stroke centers have advocated use of 6-Sigma manufacturing methods to optimize the triage process and reduce time to treatment.

Many vascular emergencies are now treated using endovascular surgery (e.g., interventional radiology, interventional cardiology, surgical interventional neuroradiology, endovascular surgical neuroradiology, endovascular surgery, embolization, catheterization lab) which uses multi-axial catheter and guidewire assemblies, such as the one depicted in FIG. 1, to perform the procedure. As is well-known and as depicted in FIG. 2, in endovascular surgery, components of a catheter and guidewire assembly (FIG. 1) are inserted into the human body through a vascular pathway. FIG. 2 depicts the arterial vascular pathway used to access the cerebral circulation for acute stroke thrombectomy and other cerebrovascular procedures; most commonly through the femoral artery at the crease in the leg. There are, however, other endovascular access pathways including the radial artery, brachial artery, axillary artery, or carotid artery.

A problem in endovascular surgery is that catheter and guidewire assemblies are typically multi-component and multi-axial assemblies that require significant, knowledgeable, and sterile assembly before use. Preparation of these devices takes time and can distract the engaged staff from other critical duties. Specific to this problem, catheters or guidewires are typically distributed by manufacturers or retailers in catheter hoops made from standard catheter hoop tubing in a sterile, hermetically sealed sheath, such as, by way of example, depicted in U.S. Pat. No. 6,053,313. The catheter hoop tubing is used to protect a catheter, guidewire, or a treatment device from heat, light, crushing, contamination, or other potential causes of damage during storage and transport. These catheters and guidewires are most often shipped as individual, stand-alone components.

When an operating physician needs catheters and guidewires for an emergency or non-emergency surgical procedure, he/she identifies all the individual components needed for the operation. The operating physician, or his/her assigns (e.g., nurses, technologists, physician assistants, or other hospital staff, etc.), then must gather from storage each individual component that the operating physician has specified for the procedure. For each component, the operating physician or assign must then open and sterilely remove each individual catheter or guidewire; prepare it for use in the operating room by hydration of the proprietary lubricating coating and by flushing each component separately of contaminants and air, using biocompatible fluid; then subsequently assemble the components, again purging any entrained air or other contaminants. Further, this flush and assembly process is critical because failure to completely remove entrained atmospheric gases or other particulates from the catheter and guidewire assemblies before their use in a patient can result in embolization of the patient's vascular system with gas or other particulates causing harm to the patient. Further still, failure to maintain catheter and guidewire systems at pressurized biocompatible fluid flush risks internal thrombosis from retrograde flow of the patient's blood components into the catheter lumens, then resulting in catheter or device malfunction or unintended iatrogenic embolization to the vascular system with blood clot.

Then, after flushing, the operating physician or assign finally assembles all the necessary, compatible components, such as catheters and guidewires, together into a multi-axial system for use in treatment. This is all very time-consuming and delays the time an operating physician can finally turn his attention to actually treating the emergency that the patient is experiencing.

Another issue caused by present catheter and guidewire packaging is that many dispenser tubes are coiled hoop tubing for compact packaging of devices. Many materials or braid-patterns used in catheters and guidewires have shape memory and can, over time, take on, and keep, the curved form of the dispenser tube, which is, for many reasons, not ideal. Any damage or deformation of catheters and guidewires caused by the coiled packaging can impair their function of the catheters and guidewires within arterial anatomy by reducing the linear vector for pushability, especially in procedures involving the cervical and cerebral circulations. As such, efforts to prevent deformation caused by packaging is desirable.

Accordingly, there is a need for multi-component medical packaging that reduces the time needed to prepare and assemble catheter and guidewire assemblies for use in surgical situations, which is no more susceptible to contamination than current methods and protects the catheter and guidewire components during transport and storage.

### SUMMARY

According to one aspect of the present invention, a multi-component medical packaging device may include several concentric tubes; where each pairing of concentric tubes forms a channel between them and where each channel is configured to house a separate medical component; and may include a fluid housing having a fluid reservoir and a fluid connection interface; where the fluid housing is integrally connected to one end of the concentric tubes, such that the channels formed by the concentric tubes are in fluid communication with the fluid reservoir and where the fluid connection interface is in fluid communication with the fluid reservoir.

According to another aspect of the present invention, the plurality of concentric tubes of the multi-component medical packaging device may be three concentric tubes which form a first channel, a second channel and a third channel, where the first channel may be configured to receive a guidewire, the second channel may be configured to receive a microcatheter and the third channel may be configured to receive an intermediate catheter. Further, the first channel may be configured to receive a guidewire having a diameter in a range from 0.014 inches to 0.018 inches and having a length in a range from 180 centimeters to 200 centimeters; the second channel may be configured to receive a microcatheter having a diameter in a range from 0.016 inches to 0.021 inches and having a length of approximately 150 centimeters; and the third channel may be configured to receive an intermediate catheter having a diameter in a range from 0.060 inches to 0.088 inches and having a length in a range from 115 centimeters to 125 centimeters.

The plurality of concentric tubes of the multi-component medical packaging device may also be disposed within a tubular outer shell that is also integrally connected to the fluid housing, and they may be chamfered at the end opposite the fluid housing. The fluid housing may include a window, and the fluid connection interface of the fluid housing may be a Luer-Lok™.

According to another aspect of the present invention, a method for selecting and protecting a catheter and guidewire assembly prior to use in surgery may include selecting a guidewire and a catheter for use in a surgery; providing a multi-component medical packaging device having at least two concentric tubes; where the at least two concentric tubes form at least a first channel and at least a second channel, and having a fluid housing that includes a fluid reservoir and a fluid connection interface; where the fluid housing is integrally connected to the at least two concentric tubes, such that the channels formed by the at least two concentric tubes are in fluid communication with the fluid reservoir and where the fluid connection interface is in fluid communication with the fluid reservoir; inserting the selected guidewire into the first channel; inserting the selected catheter into the second channel; securing the selected guidewire and the selected catheter in their respective first and second channels to form a multi-axial catheter and guidewire assembly; and sealing the multi-axial catheter and guidewire assembly in a contaminant protection sleeve for transport and handling.

Further, according to other aspects of the present invention, where the step of selecting at least one guidewire and at least one catheter for use in a surgery may be done through an online website. Also, the selected at least one guidewire may be a microguidewire, the selected at least one catheter may be a microcatheter and the provided multi-component medical packaging device may have a third concentric tube which forms a third channel, in addition to the first channel and the second channel, which may further include selecting a second catheter, which is an intermediate catheter, for use in a surgery; inserting the selected second catheter into the third channel; and in addition to securing the selected microguidewire and the selected microcatheter in their respective first and second channels, securing the selected intermediate catheter in the third channel to form the multi-axial catheter and guidewire assembly for sealing.

The method of an aspect of the present invention may further include connecting a rotating hemostatic valve to the microcatheter prior to inserting the microcatheter into a second channel; and connecting a rotating hemostatic valve to the intermediate catheter prior to inserting the intermediate catheter into a third channel. The method may also include, at the step of securing the selected microguidewire, the selected first microcatheter and the selected intermediate catheter in their respective first, second and third channels, providing a torque device which is connected to the microguidewire to secure the selected microguidewire, the selected first microcatheter and the selected intermediate catheter in their respective first, second and third channels. The method may further include providing a retaining member; placing the retaining member around the rotating hemostatic valve connected to the microcatheter; the rotating hemostatic valve connected to the intermediate catheter; the torque device and portions of the microguidewire, the microcatheter and the intermediate catheter disposed outside of the multi-component medical packaging device to create a secure assembly; placing the secure assembly in a protective molded wrapper to create a wrapped secure assembly; where, at the step of sealing the multi-axial catheter and guidewire assembly for transport and handling: placing the wrapped secure assembly into a contaminant protection sleeve; and sterilizing and sealing the contaminant protection sleeve. Further, the provided retaining member may have a serrated edge.

According to another aspect of the present invention, a method for preparing a catheter and guidewire assembly for surgery may include providing a multi-axial catheter and guidewire assembly, having a guidewire; a first catheter having a rotating hemostatic valve attached thereto; a second catheter having a rotating hemostatic valve attached thereto; a torque device; a retaining member; a multi-component medical device having at least three concentric tubes; wherein the at least three concentric tubes form a first channel, a second channel and a third channel where the guidewire may be disposed in the first channel, the first catheter may be disposed in the second channel and the second catheter may be disposed in the third channel; and having a fluid housing that includes a fluid reservoir and a fluid connection interface; wherein the fluid housing is integrally connected to the at least three concentric tubes, such that the channels formed by the at least three concentric tubes are in fluid communication with the fluid reservoir and wherein the fluid connection interface is in fluid communication with the fluid reservoir; and where the torque device is connected to the guidewire to secure the guidewire and the first and second catheters in their respective first, second and third channels and where the retaining member is in place around the rotating hemostatic valve connected to the first catheter; the rotating hemostatic valve connected to the second catheter; the torque device and portions of the guidewire and the first and second catheters disposed outside of the multi-component medical packaging device; providing a fluid flush assembly having tubing and a fluid bag filled with a sterilization fluid; connecting the tubing of the fluid flush assembly to the fluid connection interface of the multi-axial catheter and guidewire assembly; filling the fluid reservoir and the first, second and third channels with the sterilization fluid until the fluid reservoir is filled; once the fluid reservoir is filled, turning the multi-axial catheter and guidewire assembly to a vertical, upright position; continuing to fill the first, second and third channels; the first catheter and its attached rotating hemostatic valve and the second catheter and its attached rotating hemostatic valve with sterilization fluid until gas and any particulates are purged from the multi-axial catheter and guidewire assembly; grasping the retaining member; pulling the retaining member apart from the multi-component medical device to remove the guidewire, the first catheter and the second catheter from the multi-component medical device; and removing the retaining member, putting the guidewire, the first catheter and the second catheter in a state to be connected to separate fluid flush assemblies and ready for use in surgery.

Further, the guidewire of the method of this aspect of the present invention may be a microguidewire; the first catheter may be a microcatheter; and the second catheter may be an intermediate catheter. The first channel may be configured to receive a guidewire having a diameter in a range from 0.014 inches to 0.018 inches and having a length in a range from 180 centimeters to 200 centimeters; the second channel may be configured to receive a microcatheter having a diameter in a range from 0.016 inches to 0.021 inches and having a length of approximately 150 centimeters; and the third channel may be configured to receive an intermediate catheter having a diameter in a range from 0.060 inches to 0.088 inches and having a length in a range from 115 centimeters to 125 centimeters. The fluid housing may include a window, and the fluid connection interface of the fluid housing may be a Luer-Lok™. The provided retaining member may have a serrated edge.

### DRAWINGS

Objects, features, and advantages of the present invention will become apparent upon reading the following description in conjunction with the drawing figures, in which:

FIG. 1 is a sectional view of an exemplary multi-axial catheter and guidewire assembly used in endovascular surgeries;

FIG. 2 depicts the most common, femoral arterial vascular pathway used to access the cerebral circulation for acute stroke thrombectomy and other cerebrovascular procedures;

FIG. 3A is a perspective view of an embodiment of a multi-component medical packaging device of the present invention;

FIG. 3B is an enlarged view of the designated portion of FIG. 3A;

FIG. 4A is a partial side view an embodiment of the multi-component medical packaging device of the present invention;

FIG. 4B is a sectional view of FIG. 4A;

FIG. 4C is an enlarged view of the designated portion of FIG. 4B;

FIG. 5A is a sectional view of an embodiment of the multi-component medical packaging device of the present invention with an exemplary guidewire inserted;

FIG. 5B is a sectional view of an embodiment of the multi-component medical packaging device of the present invention with an exemplary guidewire and intermediate catheter assembly inserted;

FIG. 5C is a sectional view of an embodiment of the multi-component medical packaging device of the present invention with an exemplary guidewire, intermediate catheter assembly and microcatheter assembly inserted;

FIG. 5D is a sectional view of an embodiment of the multi-component medical packaging device of the present invention with an exemplary complete multi-axial catheter and guidewire assembly inserted;

FIG. 6 is an enlarged view of the designated portion of FIG. 5D;

FIG. 7A is a side view of an embodiment of the multi-component medical packaging device of the present invention with an exemplary complete multi-axial catheter and guidewire assembly inserted and a retaining member in place;

FIG. 7B is a side view of an embodiment of the multi-component medical packaging device of the present invention with an exemplary complete multi-axial catheter and guidewire assembly inserted and a retaining member in place within a protective molded wrapper;

FIG. 7C is a side view of an embodiment of the multi-component medical packaging device of the present invention with an exemplary complete multi-axial catheter and guidewire assembly inserted and a retaining member in place within a protective molded wrapper and within a sleeve;

FIG. 8 is a side view of the sleeve, containing an embodiment of the multi-component medical packaging device of the present invention with an exemplary complete multi-axial catheter and guidewire assembly inserted and a retaining member in place within a protective molded wrapper, being opened;

FIG. 9A is a side view of an embodiment of the multi-component medical packaging device of the present invention with an exemplary complete multi-axial catheter and guidewire assembly inserted and a retaining member in place connected to a fluid flush assembly with fluid at a first level;

FIG. 9B is a side view of an embodiment of the multi-component medical packaging device of the present invention with an exemplary complete multi-axial catheter and guidewire assembly inserted and a retaining member in place connected to a fluid flush assembly with fluid at a second level;

FIG. 9C is a side view of an embodiment of the multi-component medical packaging device of the present invention with an exemplary complete multi-axial catheter and guidewire assembly inserted and a retaining member in place connected to a fluid flush assembly with fluid at a third level;

FIG. 9D is a side view of an embodiment of the multi-component medical packaging device of the present invention with an exemplary complete multi-axial catheter and guidewire assembly inserted and a retaining member in place connected to a fluid flush assembly with the entire assembly in a vertical position and the fluid at a fourth level;

FIG. 9E is a sectional view of the designated portion of FIG. 9D;

FIG. 9F is a side view of an embodiment of the multi-component medical packaging device of the present invention with an exemplary complete multi-axial catheter and guidewire assembly inserted and a retaining member in place connected to a fluid flush assembly with the entire assembly in a vertical position and the fluid at a fifth level;

FIG. 9G is a side view of an embodiment of the multi-component medical packaging device of the present invention with an exemplary complete multi-axial catheter and guidewire assembly inserted and a retaining member in place connected to a fluid flush assembly with the entire assembly in a vertical position and the fluid at a sixth level;

FIG. 9H is a side view of an embodiment of the multi-component medical packaging device of the present invention with an exemplary complete multi-axial catheter and guidewire assembly inserted and a retaining member in place with the fluid filling the openings withing the entire assembly; and

FIG. 10 is an illustration of the complete multi-axial catheter and guidewire assembly connected to fluid flush assemblies and ready for use.

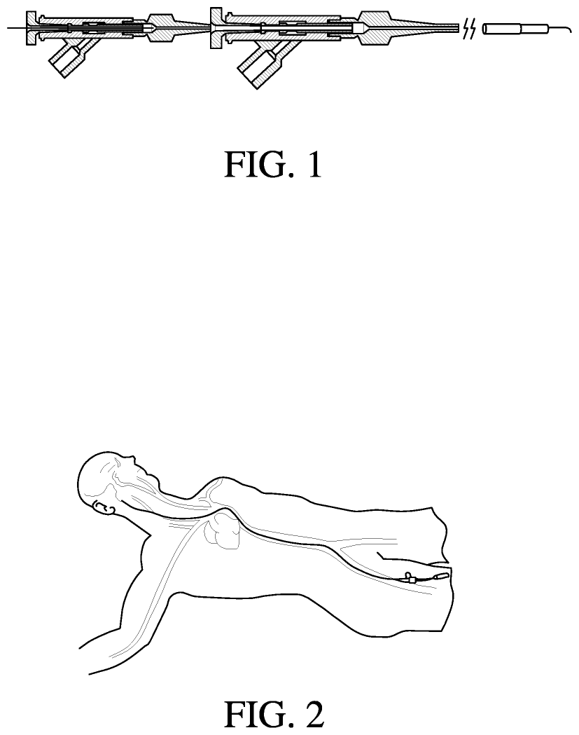

### DESCRIPTION

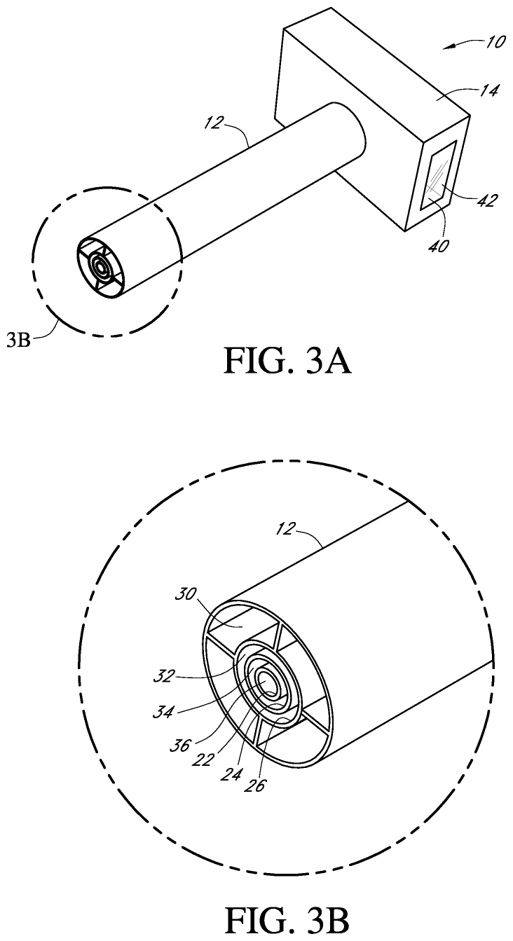

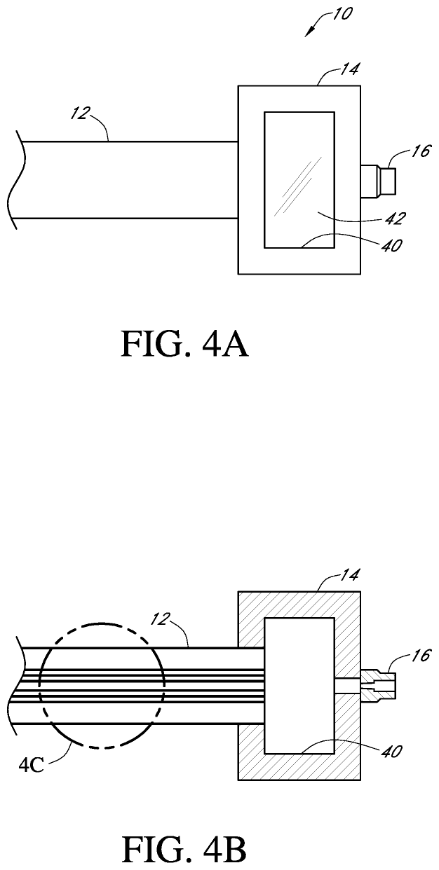

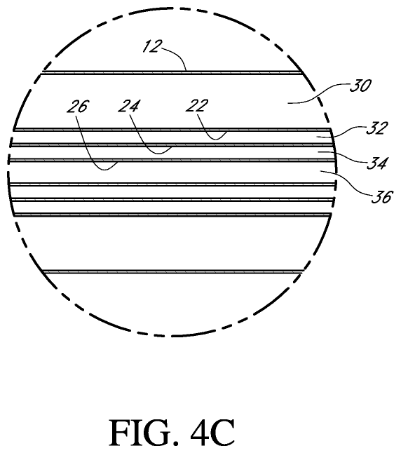

Referring to FIGS. 3A-3B and FIGS. 4A-4C, an embodiment of a multi-component medical packaging device 10 of the present invention is depicted. The multi-component medical packaging device 10 includes a tubular outer shell 12, a fluid housing 14 and a fluid connection interface 16 (FIG. 4A). Further, as depicted in FIG. 3B, within the outer shell 12, the multi-component medical packaging device 10 includes concentric tubes 22, 24, 26 that form channels 30, 32, 34, 36 (FIGS. 3B, 4C). In this embodiment, the edges of concentric tubes 22, 24, 26, at the open end (i.e. the end opposite the fluid housing 14) are beveled, or chamfered, to prevent damage to equipment inserted in the concentric tubes 22, 24, 26 (e.g., to prevent gouging of the catheter or guidewire coatings). Also, in this embodiment, the outer shell 12 and the concentric tubes 22, 24, 26 are integrated with the fluid housing 14 (FIG. 4B), and the outer shell 12 and the concentric tubes 22, 24, 26 are separately molded and welded using chemical or thermal means, extruded, formed from nitinol hypotubes, or printed using thermoplastic computer-aided design (CAD). It is envisioned that in other embodiments, the outer shell 12 and the concentric tubes 22, 24, 26 can be formed or made in any manner that is workable for the purposes of the invention and may be formed to be separable from the fluid housing 14. Also, in this embodiment, the concentric tubes 22, 24, 26 have increasing diameters from the innermost tube 22 to the outermost tube 26. Further, it should be understood that three concentric tubes 22, 24, 26 are depicted for this embodiment, but that any number of concentric tubes may be formed within the outer shell 12, as needed.

Referring specifically to FIGS. 4A and 4B, the fluid housing 14 includes a fluid reservoir 40 and a window 42, which allows an outside observer to see into the fluid reservoir 40. The fluid reservoir 40 is in fluid communication with the fluid connection interface 16 on one side and with the interior of the outer shell 12, including the channels 30, 32, 34, 36, on the other side. In one embodiment, the fluid connection interface 16 is a Luer-Lok™. Further, the fluid connection interface 16, in addition to other variants, may be a threaded or non-threaded collar.

When a surgeon is preparing for surgery, the surgeon, assign, or other member of his staff plans for the surgery and decides what catheters and guidewires the surgeon wants or needs for that specific endovascular procedure, as is the present practice. The surgeon, assign, or other member of his staff then selects those components. In the past, as described above, the surgeon, assign, or other member of his staff would have to undertake the time-consuming process of retrieving each needed surgical component and prepping each component for surgery. With the multi-component medical packaging device 10 of the present invention, the surgeon can have all the selected catheters and guidewires for a specific surgery pre-loaded in one package, and this package can be used to prep the selected catheters and guidewires for use in surgery at the same time.

In one embodiment, the surgeon, assign, or other member of his staff notifies a packager of the selected components to be packaged in the multi-component medical packaging device 10. It is envisioned that the surgeon could do the component selection and notification in one of several ways, including a selection process through a website online. The packager, once it receives the surgeon's request, pre-loads the requested components into the multi-component packaging device 10 for subsequent sterilization and shipping.

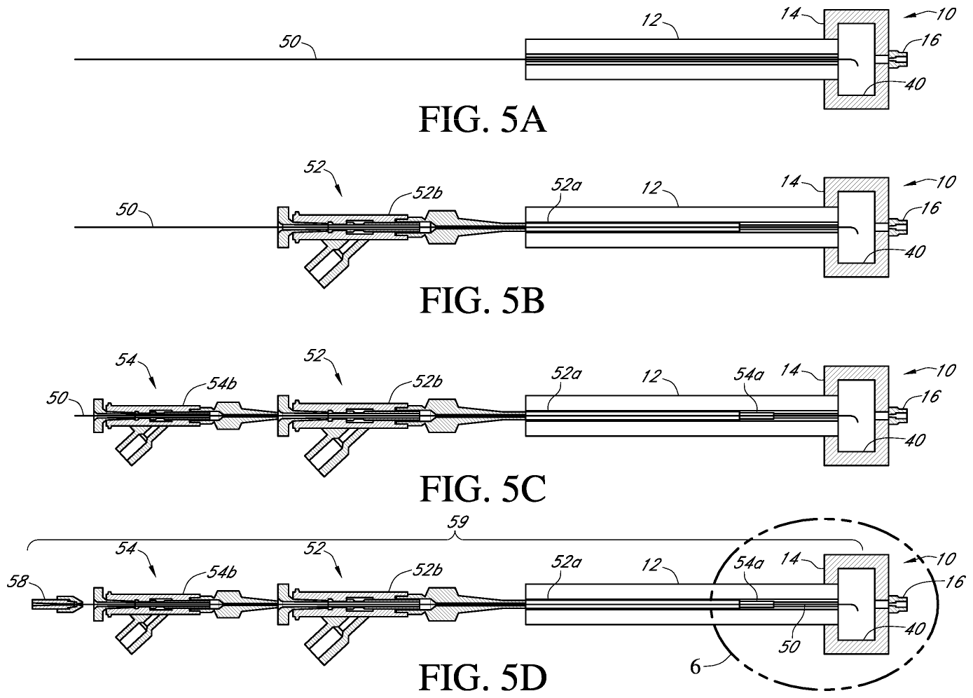

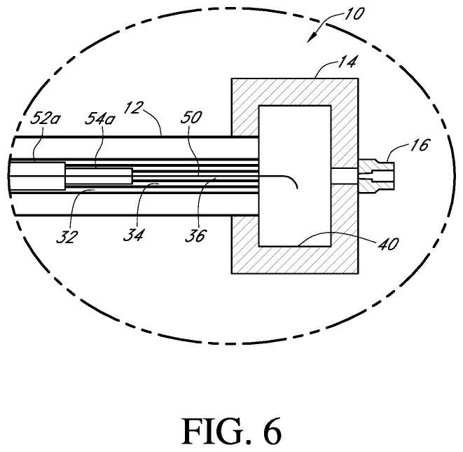

Referring to FIGS. 5A-5D, 6, an example of pre-loading the multi-component medical packaging device 10 of the present invention is depicted. In this example, the surgeon has selected one microguidewire, one microcatheter and one intermediate catheter for the acute stroke procedure the surgeon plans on performing. An intermediate catheter may be any catheter that supports the intended procedure occurring distally from the surgeon; including, among others, support catheters, suction catheters and port catheters. For an acute stroke procedure, the microguidewires commonly used range in diameter from 0.014-0.018 inches and range in length from 180-200 centimeters; the microcatheters commonly used range in diameter from 0.016-0.021 inches and typically have a length of 150 centimeters; and the intermediate or aspiration catheters commonly used range in diameter from 0.060-0.088 inches and range in length from 115-125 centimeters. After selecting these components, the surgeon, assign, or other member of his staff then notifies the packager of the selected components. In this example, the packager then retrieves the selected microguidewire 50, the selected microcatheter assembly 54 and the selected intermediate catheter assembly 52. In some embodiments, it is envisioned that the packager will source the individual selected components from different manufacturers, then extract each selected component from its original, individual packaging in a controlled environment (usually a clean room) to prepare the selected components for re-packaging in the multi-component packaging device 10.

With the selected components retrieved, prepped and ready for packaging in the multi-component packaging device 10, the packager, in a clean room meeting ISO standards, proceeds to insert the selected components into the multi-component packaging device 10. In this example, referring to FIGS. 5A, 6, the packager first inserts the microguidewire 50 into the innermost channel 36 of the multi-component packaging device 10. Next, as depicted in FIGS. 5B, 6, the packager threads a rotating hemostatic valve 52b (aka a Tuohy-Borst adapter) into connection with an intermediate catheter 52a to create the intermediate catheter assembly 52. The packager then places the intermediate catheter assembly 52 over the microguidewire 50; moves the intermediate catheter assembly 52 forward over the microguidewire 50 until the intermediate catheter 52a is inserted into the channel 32 of the multi-component packaging device 10. Next, as depicted in FIGS. 5C, 6, the packager threads a rotating hemostatic valve 54b into connection with a microcatheter 54a to create the microcatheter assembly 54. The packager then places the microcatheter assembly 54 over the microguidewire 50; moves the microcatheter assembly 54 forward over the microguidewire 50 and pushes the microcatheter 54a into intermediate catheter assembly 52 and continues to push the microcatheter 54a forward until it is inserted into the middle channel 34 of the multi-component packaging device 10. With the selected microguidewire 50, the selected microcatheter assembly 54 and the selected intermediate catheter assembly 52 in place in the multi-component packaging device 10, the packager then, as depicted in FIG. 5D, threads a torque device 58 on to the end of the microguidewire 50 to secure all the components in place to create a complete multi-axial catheter and guidewire assembly 59.

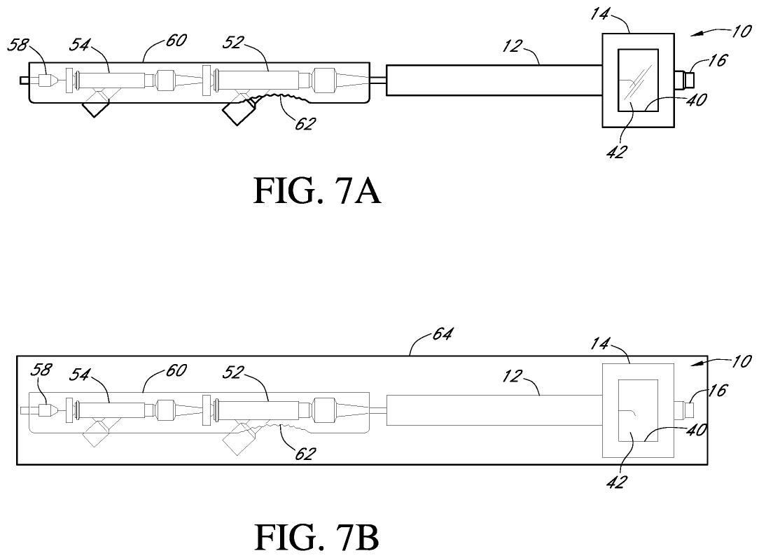

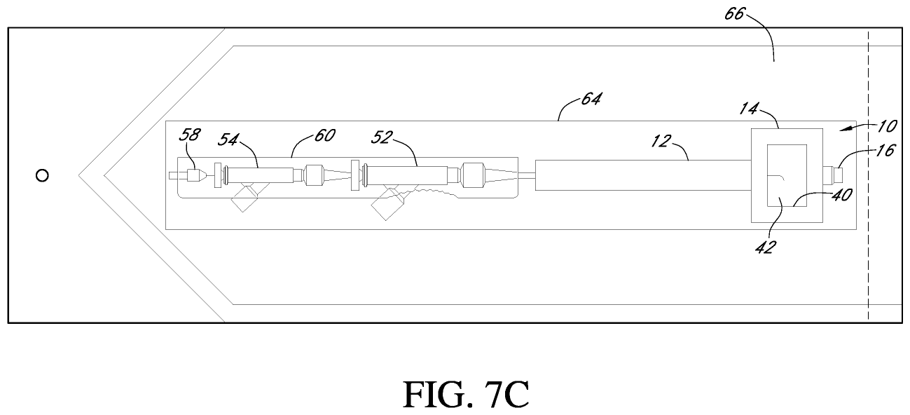

Referring now to FIGS. 7A-C, with the multi-component packaging device 10 pre-loaded with the selected microguidewire 50, the selected microcatheter assembly 54 and the selected intermediate catheter assembly 52, the packager finishes packaging the multi-component packaging device 10 for shipment and use by the surgeon who selected the components. As depicted in FIG. 7A, the packager places a retaining member 60 around the portions of the selected components that are not contained within the multi-component packaging device 10 to secure these portions of the selected components in place during transport and handling. In this embodiment, the retaining member 60 is molded to the shape of the portions of the selected components that are not contained within the multi-component packaging device 10, and the retaining member 60 has a serrated edge 62 formed thereon that, as described below, is used by the surgeon, assign, or other member of his staff to ease removal of the microguidewire 50, the microcatheter assembly 54 and the intermediate catheter assembly 52 from the multi-component packaging device 10 after hydration and preparation for use.

Next, as depicted in FIG. 7B, in this embodiment, the packager places the combined pre-loaded multi-component packaging device 10 and retaining member 60 into a protective molded wrapper 64, typically plastic, to secure the components in place and protect them during transport and handling from premature separation. Finally, as depicted in FIG. 7C, the packager places the wrapped combined pre-loaded multi-component packaging device 10 and retaining member 60 into a contaminant protection sleeve 66, which in this embodiment is made from cellophane and Tyvek® which makes the sleeve 66 permeable to gas, but not permeable to contaminants. The packager then subjects the combined pre-loaded multi-component packaging device 10 and retaining member 60 in the sleeve 66 to gas sterilization, and hermetically seals the sleeve 66. Hermetically sealing the sleeve 66 keeps the combined pre-loaded multi-component packaging device 10 and retaining member 60 free from contamination until the sleeve 66 is opened at the time of use in an operating room. The sleeve 66, containing the wrapped combined pre-loaded multi-component packaging device 10 and retaining member 60, is typically placed in a box at this point and is now ready to be sent to the surgeon who requested the selected components. To reduce medical waste, in a preferred embodiment of the invention, the packaging materials used for all the components of the invention (e.g., the multi-component packaging device 10, the retaining member 60 and the sleeve 66) are recyclable.

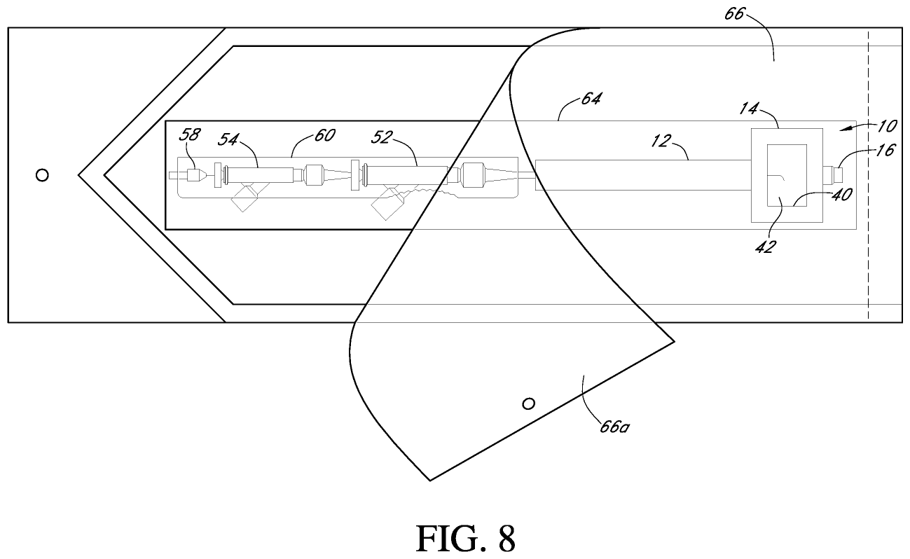

Later, when the surgeon is ready to use the complete multi-axial catheter and guidewire assembly 59, including the selected microguidewire 50, the microcatheter assembly 54 and the intermediate catheter assembly 52 in a specific surgical procedure, the surgeon, assign, or other member of his staff requests that the operating room or catheterization laboratory circulating personnel retrieve the surgery-specific sleeve 66 containing the combined pre-loaded multi-component packaging device 10 and retaining member 60 for use. Referring now to FIGS. 8, 9A-9H and 10, the steps of unpacking the selected components 50, 52, 54 from the packaging and preparing them for use in surgery are depicted. As depicted in FIG. 8, the circulating person preparing the components first opens the hermetically sealed sleeve 66 by pulling open a top layer 66a of the sleeve 66. This now gives medical personnel access to the wrapped, combined pre-loaded multi-component packaging device 10 and retaining member 60. Wearing sterilized gown and gloves, a component preparer (e.g., an operating assistant or a physician) reaches into the opened sleeve 66, grasps the wrapped, combined pre-loaded multi-component packaging device 10 and retaining member 60 and removes the assembly 10,60 from the sleeve 66. The component preparer then removes the outer wrapper 64.

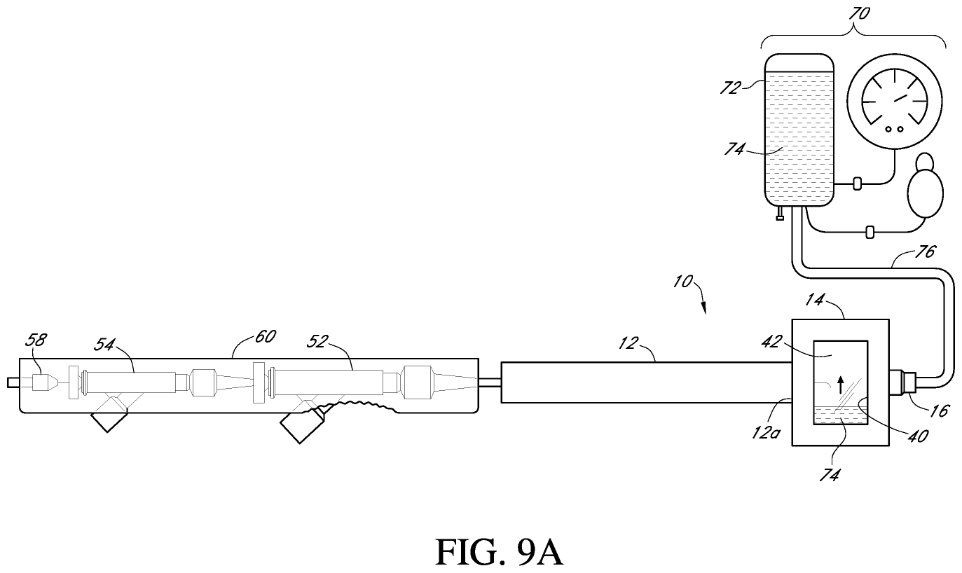

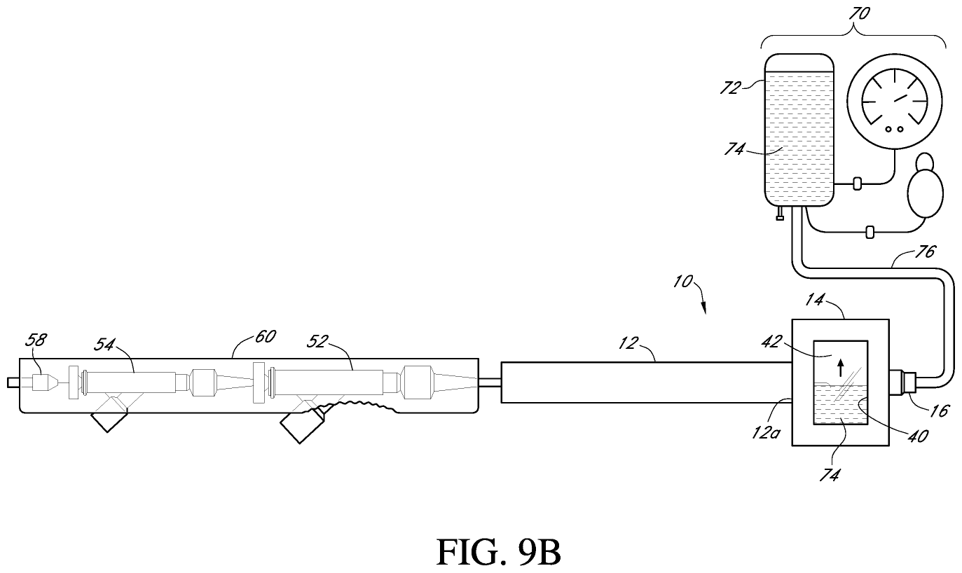

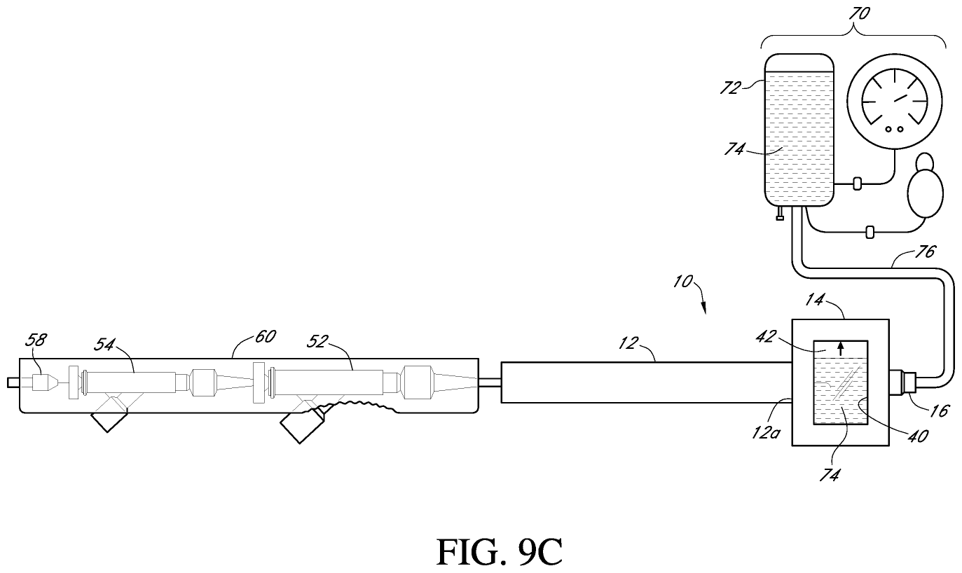

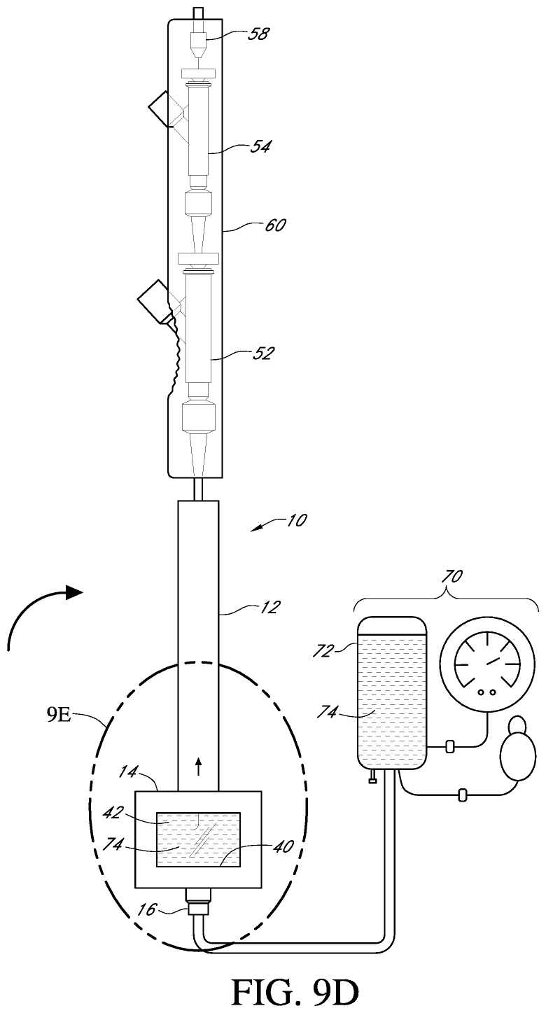

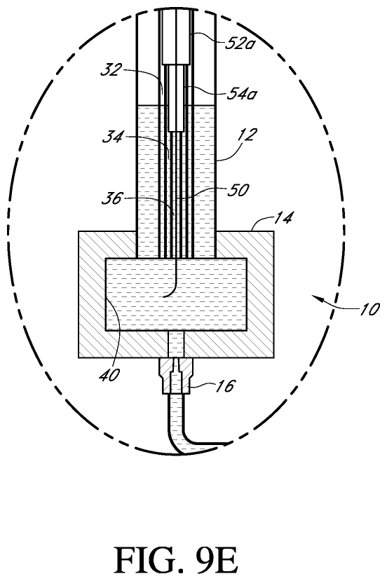

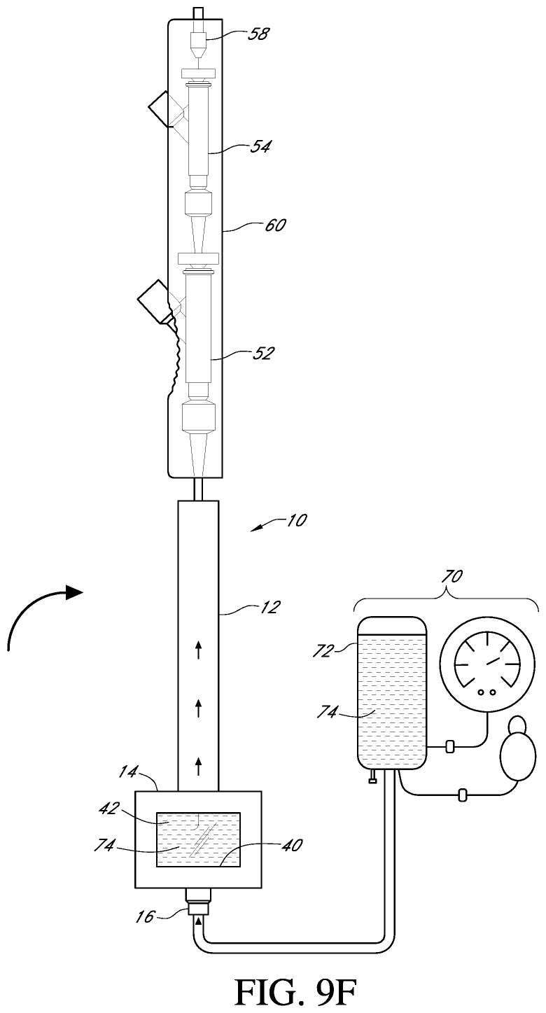

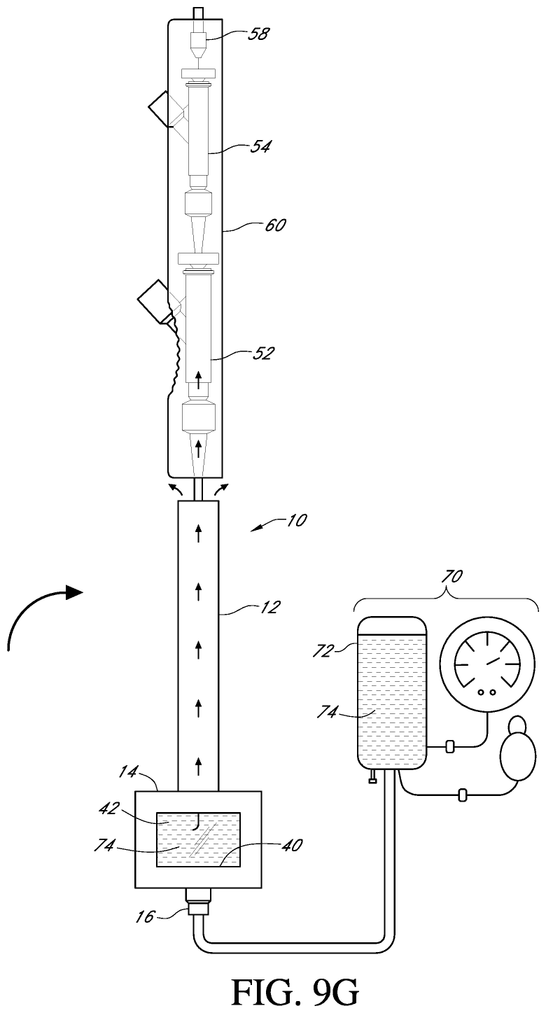

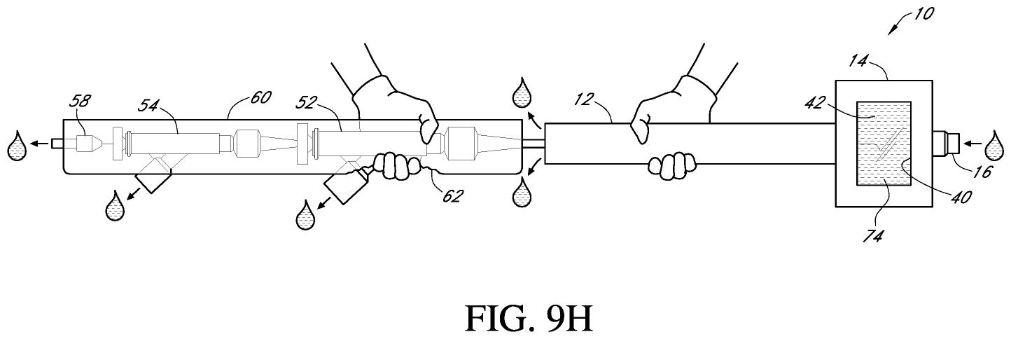

Next, as depicted in FIG. 9A, in this embodiment, to prepare the components 50, 52, 54 for use, the component preparer places the pre-loaded multi-component packaging device 10 initially horizontally on an operating table or other preparation surface. The component preparer then selects a pressurized external fluid flush assembly 70. The fluid in the fluid flush assembly 70 may be normal saline (i.e., “saline” or 0.9% sodium chloride solution), sterile Lactated Ringer's solution, or some other biocompatible fluid. The fluid flush assembly 70 includes tubing 76 and a fluid bag 72, which can be filled with a fluid 74. The component preparer connects the tubing 76 to the fluid connection interface 16 of the multi-component packaging device 10. The tubing 76 can be connected to the fluid connection interface 16 through any means known in the art; in this embodiment, it is done using a Luer-Lok™. Once the tubing 76 is connected, the pressurized fluid 74 starts to fill the fluid reservoir 40 of the multi-component packaging device 10 through the fluid connection interface 16. The translucent window 42 allows the component preparer to observe the level of the fluid 74 as it fills the reservoir 40 under super-physiological pressure (150-300 mm Hg or 29-58 psi). As the fluid 74 fills the reservoir 40 above a lower lip 12a of the outer shell 12, as depicted in FIG. 9B, the fluid 74 starts to fill the outer channel 30 in fluid communication with the fluid reservoir 40, and as depicted in FIG. 9C, as the fluid 74 continues to rise in the fluid reservoir 40, the fluid 74 fills the channels 32, 34, 36 (FIGS. 3B, 4C) as well. As the fluid 74 fills the channels 30, 32, 34, 36, the fluid 74 is purging each component 50, 52, 54 of the pre-loaded multi-component packaging device 10.

In this embodiment, as illustrated in FIG. 9D, once the fluid reservoir 40 is full, the component preparer turns the pre-loaded multi-component packaging device 10 to a vertical, upright position. As illustrated in FIGS. 9D-9E, the pressurized fluid 74 continues to move upward; continuing to fill the channels 30, 32, 34, 36 with saline. As depicted in FIGS. 9F-9H, the pressurized fluid 74 continues in this manner until the fluid 74 fills and exits every open orifice of the multi-component packaging device 10, the intermediate catheter assembly 52 and the microcatheter assembly 54 (FIG. 9H). This is done to purge gas and any particulates from the multi-component packaging device 10, the intermediate catheter assembly 52 and the microcatheter assembly 54. This also serves to hydrate the lubricating coatings on each component 50, 52, 54 to reduce each component's friction to prepare the components 50, 52, 54 for use in a human.

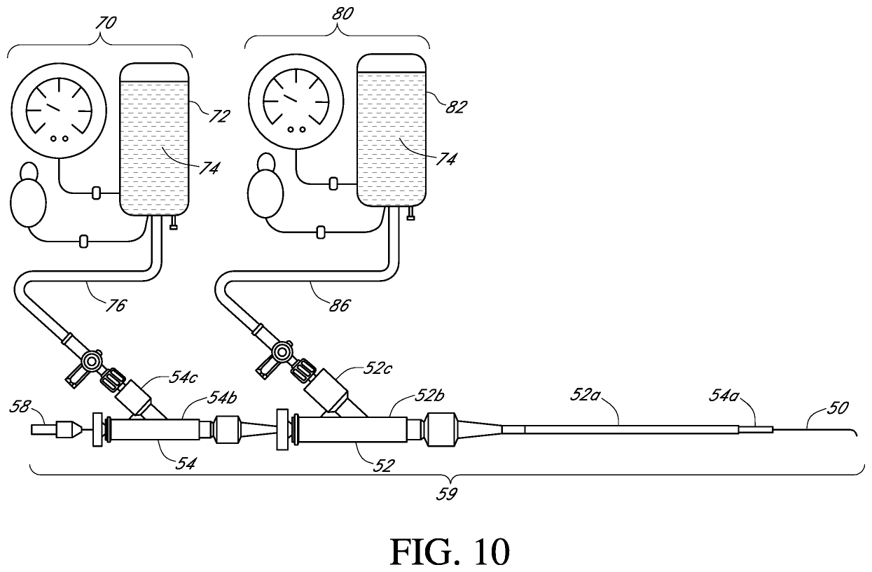

After completion of the fluid purge, as depicted in FIG. 9H, the complete multi-axial catheter and guidewire assembly 59 can be removed intact as a single unit from the multi-component packaging device 10. In this embodiment, to do that, the component preparer, while wearing gloves, grasps the retaining member 60 with one hand and grasps the multi-component packaging device 10 with the other hand and pulls the retaining member 60 apart from the multi-component packaging device 10. In this embodiment, the component preparer will feel mild traction due to the disengagement of the components 50, 52, 54 from the multi-component packaging device 10. As depicted in FIG. 10, once the complete multi-axial catheter and guidewire assembly 59 is fully removed from the multi-component packaging device 10, the component preparer, at the operator's discretion or according to institutional standards, connects each of the exit ports 52c, 54c of the catheters 52, 54 to their own separate, fluid flush assemblies 70, 80. With the complete multi-axial catheter and guidewire assembly 59 prepared in this manner, the complete multi-axial catheter and guidewire assembly 59 is ready for immediate application in urgent or emergent patient care.

With the advancement of robotic assistants in the operating room, in another embodiment, the pre-loaded multi-component packaging device 10 of FIG. 7A is configured for ease of use by a robotic assistant. In this embodiment, the pre-loaded multi-component packaging device 10 is configured as a cartridge that a robotic assistant can remove from the sleeve 66 and then perform the hydrating, lubricating and purging actions to prepare the complete multi-axial catheter and guidewire assembly 59 for connection to the fluid flush assemblies 70, 80, as depicted in FIG. 10.

Although certain embodiments and features of a multi-component medical packaging device have been described herein, the scope of coverage of this patent is not limited thereto. On the contrary, this patent covers all embodiments of the teachings of the disclosure that fairly fall within the scope of permissible equivalents.

What is claimed is:

1. A multi-component medical packaging device, comprising:
a plurality of concentric tubes; wherein each pairing of concentric tubes forms a channel therebetween and wherein each channel is configured to house a separate medical component; and
a fluid housing having a fluid reservoir and a fluid connection interface; wherein the fluid housing is integrally connected to one end of the plurality of concentric tubes, such that the channels formed by the plurality of concentric tubes are in fluid communication with the fluid reservoir and wherein the fluid connection interface is in fluid communication with the fluid reservoir.

2. The multi-component medical packaging device of claim 1, wherein the plurality of concentric tubes is three concentric tubes which form a first channel, a second channel and a third channel, wherein the first channel is configured to receive a guidewire, the second channel is configured to receive a microcatheter and the third channel is configured to receive an intermediate catheter.

3. The multi-component medical packaging device of claim 2, wherein:
the first channel is configured to receive a guidewire having a diameter in a range from 0.014 inches to 0.018 inches and having a length in a range from 180 centimeters to 200 centimeters;
the second channel is configured to receive a microcatheter having a diameter in a range from 0.016 inches to 0.021 inches and having a length of approximately 150 centimeters;
and
the third channel is configured to receive an intermediate catheter having a diameter in a range from 0.060 inches to 0.088 inches and having a length in a range from 115 centimeters to 125 centimeters.

4. The multi-component medical packaging device of claim 1, wherein the plurality of concentric tubes are disposed within a tubular outer shell that is also integrally connected to the fluid housing.

5. The multi-component medical packaging device of claim 1, wherein the fluid housing further includes a window.

6. The multi-component medical packaging device of claim 1, wherein the fluid connection interface of the fluid housing is a Luer-Lok™.

7. The multi-component medical packaging device of claim 1, wherein the plurality of concentric tubes are chamfered at the end opposite the fluid housing.

8. A method for selecting and protecting a catheter and guidewire assembly prior to use in surgery, comprising:
selecting at least one guidewire and at least one catheter for use in a surgery;
providing a multi-component medical packaging device having at least two concentric tubes; wherein the at least two concentric tubes form at least a first channel and at least a second channel, and having a fluid housing that includes a fluid reservoir and a fluid connection interface; wherein the fluid housing is integrally connected to the at least two concentric tubes, such that the channels formed by the at least two concentric tubes are in fluid communication with the fluid reservoir and wherein the fluid connection interface is in fluid communication with the fluid reservoir;
inserting the selected guidewire into the first channel;
inserting the selected catheter into the second channel;
securing the selected guidewire and the selected catheter in their respective first and second channels to form a multi-axial catheter and guidewire assembly; and
sealing the multi-axial catheter and guidewire assembly in a contaminant protection sleeve for transport and handling.

9. The method for selecting and protecting a catheter and guidewire assembly prior to use in surgery of claim 8, wherein the step of selecting at least one guidewire and at least one catheter for use in a surgery is through an online website.

10. The method for selecting and protecting a catheter and guidewire assembly prior to use in surgery of claim 8, wherein:
the selected at least one guidewire is a microguidewire, the selected at least one catheter is a microcatheter and the provided multi-component medical packaging device has a third concentric tube which forms a third channel, in addition to the first channel and the second channel, further comprising:
selecting a second catheter, which is an intermediate catheter, for use in a surgery;
inserting the selected second catheter into the third channel; and
in addition to securing the selected microguidewire and the selected microcatheter in their respective first and second channels, securing the selected intermediate catheter in the third channel to form the multi-axial catheter and guidewire assembly for sealing.

11. The method for selecting and protecting a catheter and guidewire assembly prior to use in surgery of claim 10, wherein:
the microguidewire inserted into the first channel has a diameter in a range from 0.014 inches to 0.018 inches and has a length in a range from 180 centimeters to 200 centimeters;
the microcatheter inserted into the second channel has a diameter in a range from 0.016 inches to 0.021 inches and has a length of approximately 150 centimeters;
and
the intermediate catheter inserted into the third channel has a diameter in a range from 0.060 inches to 0.088 inches and has a length in a range from 115 centimeters to 125 centimeters.

12. The method for selecting and protecting a catheter and guidewire assembly prior to use in surgery of claim 11, further comprising:
connecting a rotating hemostatic valve to the microcatheter prior to inserting the microcatheter into the second channel; and
connecting a rotating hemostatic valve to the intermediate catheter prior to inserting the intermediate catheter into the third channel.

13. The method for selecting and protecting a catheter and guidewire assembly prior to use in surgery of claim 12, wherein, at the step of securing the selected microguidewire, the selected first microcatheter and the selected intermediate catheter in their respective first, second and third channels, a torque device is provided and connected to the microguidewire to secure the selected microguidewire, the selected first microcatheter and the selected intermediate catheter in their respective first, second and third channels.

14. The method for selecting and protecting a catheter and guidewire assembly prior to use in surgery of claim 13, further comprising:
providing a retaining member;
placing the retaining member around the rotating hemostatic valve connected to the microcatheter; the rotating hemostatic valve connected to the intermediate catheter; the torque device and portions of the microguidewire, the microcatheter and the intermediate catheter disposed outside of the multi-component medical packaging device to create a secure assembly;
placing the secure assembly in a protective molded wrapper to create a wrapped secure assembly;
wherein, at the step of sealing the multi-axial catheter and guidewire assembly for transport and handling:
placing the wrapped secure assembly into a contaminant protection sleeve; and
sterilizing and sealing the contaminant protection sleeve.

15. The method for selecting and protecting a catheter and guidewire assembly prior to use in surgery of claim 14, wherein the provided retaining member has a serrated edge.

16. A method for preparing a catheter and guidewire assembly for surgery, comprising:
providing a multi-axial catheter and guidewire assembly, having:
a guidewire;
a first catheter having a rotating hemostatic valve attached thereto;
a second catheter having a rotating hemostatic valve attached thereto;
a torque device;
a retaining member;
a multi-component medical device having at least three concentric tubes;
wherein the at least three concentric tubes form a first channel, a second channel and a third channel wherein the guidewire is disposed in the first channel, the first catheter is disposed in the second channel and the second catheter is disposed in the third channel; and having a fluid housing that includes a fluid reservoir and a fluid connection interface; wherein the fluid housing is integrally connected to the at least three concentric tubes, such that the channels formed by the at least three concentric tubes are in fluid communication with the fluid reservoir and wherein the fluid connection interface is in fluid communication with the fluid reservoir; and
wherein the torque device is connected to the guidewire to secure the guidewire and the first and second catheters in their respective first, second and third channels and wherein the retaining member is in place around the rotating hemostatic valve connected to the first catheter; the rotating hemostatic valve connected to the second catheter; the torque device and portions of the guidewire and the first and second catheters disposed outside of the multi-component medical packaging device;
providing a fluid flush assembly having tubing and a fluid bag filled with a sterilization fluid;
connecting the tubing of the fluid flush assembly to the fluid connection interface of the multi-axial catheter and guidewire assembly;
filling the fluid reservoir and the first, second and third channels with the sterilization fluid until the fluid reservoir is filled;
once the fluid reservoir is filled, turning the multi-axial catheter and guidewire assembly to a vertical, upright position;
continuing to fill the first, second and third channels; the first catheter and its attached rotating hemostatic valve and the second catheter and its attached rotating hemostatic valve with sterilization fluid until gas and any particulates are purged from the multi-axial catheter and guidewire assembly;
grasping the retaining member;
pulling the retaining member apart from the multi-component medical device to remove the guidewire, the first catheter and the second catheter from the multi-component medical device; and
removing the retaining member, putting the guidewire, the first catheter and the second catheter in a state to be connected to separate fluid flush assemblies and ready for use in surgery.

17. The method for preparing a catheter and guidewire assembly for surgery, of claim 16, wherein:
the guidewire is a microguidewire;
the first catheter is a microcatheter; and
the second catheter is an intermediate catheter.

18. The method for preparing a catheter and guidewire assembly for surgery, of claim 17, wherein:
the microguidewire inserted into the first channel has a diameter in a range from 0.014 inches to 0.018 inches and has a length in a range from 180 centimeters to 200 centimeters;
the microcatheter inserted into the second channel has a diameter in a range from 0.016 inches to 0.021 inches and has a length of approximately 150 centimeters;
and
the intermediate catheter inserted into the third channel has a diameter in a range from 0.060 inches to 0.088 inches and has a length in a range from 115 centimeters to 125 centimeters.

19. The method for preparing a catheter and guidewire assembly for surgery, of claim 16, wherein the fluid housing further includes a window.

20. The method for preparing a catheter and guidewire assembly for surgery, of claim 16, wherein the provided retaining member has a serrated edge.
